(19) United States
(12) Patent Application Publication
(10) Pub. No.: US 2021/0106549 A1
(43) Pub. Date: Apr. 15, 2021

(54) PHARMACEUTICAL COMPOSITIONS AND METHODS

(71) Applicant: TYME, INC., New York, NY (US)

(72) Inventor: Steven Hoffman, Mahwah, NJ (US)

(21) Appl. No.: 17/071,416

(22) Filed: Oct. 15, 2020

Related U.S. Application Data
(60) Provisional application No. 62/915,177, filed on Oct. 15, 2019.

(51) Int. Cl. A61K 31/216; A61K 9/00

(52) U.S. Cl. CPC A61K 31/216; A61K 45/06; A61K 9/0019

(57) ABSTRACT

Pharmaceutical compositions and kits including an alkylester of α-methyl-DL-tyrosine (or salt thereof), for example, α-methyl-DL-tyrosine methyl ester hydrochloride, are provided. Also provided are methods of treating cancer in a subject, comprising administering an effective amount of an alkylester of α-methyl-DL-tyrosine (or salt thereof), for example, α-methyl-DL-tyrosine methyl ester hydrochloride, to the subject in need thereof.

### CROSS-REFERENCE TO RELATED APPLICATION

This application claims the benefit of U.S. Provisional Patent Application No. 62/915,177, filed Oct. 15, 2019, the entirety of which is incorporated by reference herein.

### TECHNICAL FIELD

The present inventions relate generally to compositions, kits and methods for the reduction of cellular proliferation as, for example, in the treatment of cancer.

### BACKGROUND

Cancer is the second most common cause of death in the United States, behind only heart disease, and accounts for one in four deaths. It has been estimated that approximately 1600 Americans die of cancer each day. In addition to the medical, emotional and psychological costs of cancer, cancer has significant financial costs to both the individual and society.

Cancer treatments today include surgery, hormone therapy, radiation, chemotherapy, immunotherapy, targeted therapy, and combinations thereof. Surgical removal of cancer has advanced significantly; however, there remains a high chance of recurrence of the disease. Hormone therapy using drugs such as aromatase inhibitors and luteinizing hormone-releasing hormone analogs and inhibitors has been relatively effective in treating prostate and breast cancers. Radiation and the related techniques of conformal proton beam radiation therapy, stereotactic radiosurgery, stereotactic radiation therapy, intraoperative radiation therapy, chemical modifiers, and radio sensitizers are effective at killing cancerous cells, but can also kill and alter surrounding normal tissue. Chemotherapy drugs such as aminopterin, cisplatin, methotrexate, doxorubicin, daunorubicin and others alone and in combinations are effective at killing cancer cells, often by altering the DNA replication process. Biological response modifier (BRM) therapy, biologic therapy, biotherapy, or immunotherapy alter cancer cell growth or influence the natural immune response, and involve administering biologic agents to a patient such as an interferons, interleukins, and other cytokines and antibodies such as rituximab and trastuzumab and even cancer vaccines such as Sipuleucel-T.

New targeted therapies have been developed to fight cancer. These targeted therapies differ from chemotherapy because chemotherapy works by killing both cancerous and normal cells, with greater effects on the cancerous cells. Targeted therapies work by influencing the processes that control growth, division, and the spread of cancer cells and signals that cause cancer cells to die naturally. One type of targeted therapy includes growth signal inhibitors such as trastuzumab, gefitinib, imatinib, centuximab, dasatinib and nilotinib. Another type of targeted therapy includes angiogenesis inhibitors such as bevacizumab that inhibit cancers from increasing surrounding vasculature and blood supply. A final type of targeted therapy includes apoptosis-inducing drugs that are able to induce direct cancer cell death.

Although all of these treatments have been effective to one degree or another, they all have drawbacks and limitations. In addition to many of the treatments being expensive, they also are often too imprecise or the cancers are able to adapt to them and become resistant.

Thus, there is a great need for additional cancer treatments. In particular, there is a need for treatments for cancers that have become resistant to other forms of treatment.

### SUMMARY

The present invention provides compositions, combination therapies, kits, and methods for reducing undue cellular proliferation, including that associated with the treatment of cancer. In one aspect, the invention provides pharmaceutical compositions comprising at least one alkylester of α-methyl-DL-tyrosine (or a pharmaceutically acceptable salt thereof), such as α-methyl-tyrosine methyl ester hydrochloride. The invention also provides pharmaceutical compositions that further comprise at least one tyrosine hydroxylase inhibitor; at least one of melanin, a melanin promoter, or a combination thereof; at least one p450 3A4 promoter; at least one leucine aminopeptidase inhibitor; and, optionally, at least one growth hormone inhibitor. In other aspects, the invention provides kits that comprise these components together with suitable packaging. Also provided are methods of reducing cellular proliferation and/or methods of treating cancer comprising administering an effective amount of an alkylester of α-methyl-DL-tyrosine (or pharmaceutically acceptable salt thereof), for example, α-methyl-tyrosine methyl ester hydrochloride, alone or in combination with; at least one of melanin, a melanin promoter, or a combination thereof, at least one p450 3A4 promoter; at least one leucine aminopeptidase inhibitor; and, optionally, at least one growth hormone inhibitor to the subject in need thereof.

### DETAILED DESCRIPTION OF ILLUSTRATIVE EMBODIMENTS

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

The term “alkyl” refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. This condition, disease or disorder can be cancer.

As employed above and throughout the disclosure the term “effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with the particular compound, component or composition selected, the route of administration, and the ability of the components to elicit a desired result in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The term “stereoisomers” refers to compounds that have identical chemical constitution, but differ as regards the arrangement of the atoms or groups in space.

The term “administering” means either directly administering a compound or composition of the present invention, or administering a prodrug, derivative or analog which will form an equivalent amount of the active compound or substance within the body.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

The term “inhibitor” as used herein includes compounds that inhibit the expression or activity of a protein, polypeptide or enzyme and does not necessarily mean complete inhibition of expression and/or activity. Rather, the inhibition includes inhibition of the expression and/or activity of a protein, polypeptide or enzyme to an extent, and for a time, sufficient to produce the desired effect.

The term “promoter” as used herein includes compounds that promote the expression or activity of a protein, polypeptide or enzyme and does not necessarily mean complete promotion of expression and/or activity. Rather, the promotion includes promotion of the expression and/or activity of a protein, polypeptide or enzyme to an extent, and for a time, sufficient to produce the desired effect.

Alkylesters of α-methyl-DL-tyrosine of the invention can exist as either or both of their respective D and L isomer. α-Methyl-tyrosine methyl ester according to the present invention can exist as either or both of its respective D and L isomers. α-Methyl-DL-tyrosine methyl ester hydrochloride preferably is used. Alkylesters of α-methyl-DL-tyrosine (and salts thereof) can be used alone or in combination with other cancer therapeutic agents. α-Methyl-DL-tyrosine methyl ester hydrochloride can be used alone or in combination with other cancer therapeutic agents.

While not intending to be bound by any particular mechanism of operation, alkylesters of α-methyl-DL-tyrosine function by accumulating in cancer cells (as either the alkyl ester or the free acid) and preventing them from forming a coating of either lipids or hyaluronan. By preventing the cancer cells from forming a coating of either lipids or hyaluron, the cancer cells are believed to be made more accessible to oxidative stress.

In one aspect, the present invention provides combination therapies that alter the defenses of cancerous cells to oxidative stress. One class of such therapies increases free radical availability to cancerous cells. A representative subclass of such therapies involves administration of pharmaceutical compositions comprising a tyrosine hydroxylase inhibitor, melanin or a melanin promoter, a p450 3A4 promoter, a leucine aminopeptidase inhibitor, and, optionally, a growth hormone inhibitor. Another subclass involves administration of pharmaceutical compositions comprising melanin and a tyrosine hydroxylase inhibitor. Particular components of pharmaceutical compositions are described below.

The present invention also can involve the use of at least one of melanin, a melanin promoter, or a combination thereof. Thus, melanin can be used, one or more melanin promoters can be used, and both melanin and one or more melanin promoters can be used (either in separate dosage forms or in the same dosage form). Melanin promoters according to the present invention are chemical compounds that increase the production and/or the activity of melanin. Representative melanin promoters are methoxsalen and melanotan II.

In some instances, the alkylester of α-methyl-DL-tyrosine (or salt thereof) is mixed with melanin in the same dosage form. In some aspects, α-methyl-DL-tyrosine methyl ester hydrochloride is mixed with melanin in the same dosage form. In certain instances, melanin is solubilized in a solubilizing agent and then mixed with the alkylester of α-methyl-DL-tyrosine (or salt thereof), for example, α-methyl-DL-tyrosine methyl ester hydrochloride, by methods known in the art. The solubilizing agent may be removed by standard techniques, such as evaporation, drying, etc. The solubilizing agent may be a non-toxic solubilizing agent, such as hydrogen peroxide or other solubilizing agents commonly known in the art. The melanin and/or the pharmaceutical composition may be further processed to optimize the pharmaceutical composition's effect on cancer cells. In another aspect, the pharmaceutical composition may include additional active agents and/or pharmaceutical excipients.

The methods of the invention may also include administration of a p450 3A4 promoter. “Cytochrome p450 3A4” (which can be abbreviated as “p450 3A4”) is a member of the cytochrome p450 superfamily of enzymes, and is a mixed-function oxidase that is involved in the metabolism of xenobiotics in the body. It has the widest range of substrates of all of the cytochromes. The function of a p450 3A4 promoter in the pharmaceutical compositions of the invention is to increase the expression and/or the activity of p450 3A4. The increased p450 3A4 expression and/or activity is believed to reduce cortisone and estrogen levels in the patient. Additionally, the increased p450 3A4 expression and/or activity also slightly decreases blood pH, which is believed to help to preserve or enhance melanin activity. Representative p450 3A4 promoters are 5,5-diphenylhydantoin (sold commercially as, for example, Dilantin), valproic acid, and carbamazepine, which are believed to induce expression of the p450 3A4 enzyme.

The instant methods may further include administration of leucine aminopeptidase inhibitors (alternatively known as leucyl aminopeptidase inhibitors). Leucine aminopeptidases are enzymes that preferentially catalyze the hydrolysis of leucine residues at the N-terminus of peptides and/or proteins. Representative leucine aminopeptidase inhibitors are N-[(2S,3R)-3-amino-2-hydroxy-4-phenylbutyryl]-L-leucine, and rapamycin.

The present methods may also optionally include administration of a growth hormone inhibitor. Growth hormone (such as, for example, pancreatic growth hormone) induces cell replication. Representative growth hormone inhibitors are octreotide, somatostatin, and seglitide.

The methods of the invention may further include administration of D-leucine. D-leucine is a stereoisomer of the naturally occurring L-leucine, the form of leucine incorporated into polypeptides and proteins. D-leucine cannot be incorporated into polypeptides and/or proteins. The presence of D-leucine may permit the use of lower doses of leucine aminopeptidase inhibitor in a pharmaceutical composition.

Also provided herein are kits including a combination therapy that creates alterations in the defenses of cancerous cells to oxidative stress. An intended suitable kit includes a combination therapy that increases free radical availability to cancerous cells. Representative kits comprise an alkylester of α-methyl-DL-tyrosine (or salt thereof), for example, α-methyl-DL-tyrosine methyl ester hydrochloride, alone or in combination another cancer therapeutic agent and/or with melanin and/or a melanin promoter, a p450 3A4 promoter, a leucine aminopeptidase inhibitor and, optionally, a growth hormone inhibitor of the type described above, together with packaging for same. The kit can include one or more separate containers, dividers or compartments and, optionally, informational material such as instructions for administration. For example, each inhibitor or promoter (or the various combinations thereof) can be contained in a bottle, vial, or syringe, and the informational material can be contained in a plastic sleeve or packet or provided in a label. In some aspects, the kit includes a plurality (e.g., a pack) of individual containers, each containing one or more unit dosage form of a compound described herein. For example, the kit can include a plurality of syringes, ampules, foil packets, or blister packs, each containing a single unit dose of a compound described herein or any of the various combinations thereof. The containers of the kits can be air tight, waterproof (e.g., impermeable to changes in moisture or evaporation), and/or light-tight. The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device.

Methods of treating cancer in a subject also are provided, as are methods of reducing undue cellular proliferation. Such methods can include administering an effective amount of an alkylester of α-methyl-DL-tyrosine (or salt thereof), for example, α-methyl-DL-tyrosine methyl ester hydrochloride, alone or as part of a combination therapy that creates alterations in the defenses of cancerous cells to oxidative stress. Representative methods of treating cancer include administering an effective amount of an alkylester of α-methyl-DL-tyrosine (or salt thereof), for example, α-methyl-DL-tyrosine methyl ester hydrochloride, alone or as part of a combination therapy that increases free radical availability to cancerous cells. Suitable methods include administering an effective amount of an alkylester of α-methyl-DL-tyrosine (or salt thereof), for example, α-methyl-DL-tyrosine methyl ester hydrochloride, alone or with the above-noted tyrosine hydroxylase inhibitor, melanin and/or melanin promoter, p450 3A4 promoter, leucine aminopeptidase inhibitor and, optionally, growth hormone inhibitor. Other suitable methods include administering an effective amount of melanin and a tyrosine hydroxylase inhibitor.

Suitable methods include simultaneous or at least contemporaneous administration of an alkylester of α-methyl-DL-tyrosine (or salt thereof), for example, α-methyl-DL-tyrosine methyl ester hydrochloride and at least one of the melanin or a melanin promoter, p450 3A4 promoter, and leucine aminopeptidase inhibitor, at least two of them, or each of them (in each case, optionally, with a growth hormone inhibitor). The desired number of inhibitors and promoters can be provided in a single dosage form or any number of desired dosage forms, including in individual dosage forms.

Representative dosage forms include tablets, capsules, caplets, sterile aqueous or organic solutions, reconstitutable powders, elixirs, liquids, colloidal or other types of suspensions, emulsions, beads, beadlets, granules, microparticles, nanoparticles, and combinations thereof. The amount of composition administered will, of course, be dependent on the subject being treated, the subject's weight, the severity of the condition being treated, the manner of administration, and the judgment of the prescribing physician.

Administration of the alkylester of α-methyl-DL-tyrosine (or salt thereof), for example, α-methyl-DL-tyrosine methyl ester hydrochloride, can be through various routes, including orally, nasally, subcutaneously, intravenously, intramuscularly, transdermally, vaginally, rectally or in any combination thereof α-Methyl-DL-tyrosine methyl ester hydrochloride is preferably administered non-orally, including subcutaneously, intravenously, intramuscularly, or transdermally, preferably in the form of aqueous solutions. Transdermal administration can be effected by using, for example, oleic acid, 1-methyl-2-pyrrolidone, or dodecylnonaoxyethylene glycol monoether.

Administration of the melanin, promoters, and/or inhibitors can be through various routes, including orally, nasally, subcutaneously, intravenously, intramuscularly, transdermally, vaginally, rectally or in any combination thereof. Transdermal administration can be effected using, for example, oleic acid, 1-methyl-2-pyrrolidone, or dodecylnonaoxyethylene glycol monoether.

The melanin, promoters and/or inhibitors can be administered during a cycle consisting of five to seven days of administering the melanin, promoters and/or inhibitors and one to two days of not administering the melanin, promoters and/or inhibitors. The melanin, promoters and/or inhibitors can be administered over the course of at least six of said cycles. It can be desirable to administer these components about two hours between meals to facilitate uptake.

The subject to which the instant compositions are administered can be a mammal, preferably a human.

In another representative method, 60 mg of α-methyl-DL-tyrosine methyl ester hydrochloride is administered orally and, optionally, 0.25 mL of a 2 mg/mL suspension of the tyrosine derivative is administered subcutaneously; 10 mg of the methoxsalen is administered orally and 0.25 mL of a 1 mg/mL suspension of the methoxsalen is administered subcutaneously; 30 mg of the 5,5-diphenylhydantoin is administered orally; and 20 mg of the N-[2S,3R)-3-amino-2-hydroxy-4-phenylbutyryl]-L-leucine is administered orally.

Representative methods include those in which the cancer is non-small cell lung cancer. In certain embodiments, the non-small cell lung cancer is stage IV non-small cell lung cancer. In other embodiments, the cancer is ovarian cancer, breast cancer, cervical cancer, pancreatic cancer, stomach cancer, brain cancer, liver cancer, testicular cancer, leukemia, lymphoma, appendix cancer, biliary cancer, choleangiocarcinoma, colon cancer, colorectal cancer, germ cell tumor, glioma, Hodgkin's lymphoma, lung cancer, neuroblastoma, prostate cancer, renal cancer, sarcoma, thyroid cancer, tongue cancer, tonsil squamous cell carcinoma, or urothelial cancer. Progression of said cancer in said subject can be assessed.

The present methods can include not only the disclosed administration step but also the step of assessing progression of said cancer in said subject and/or the extent of cellular proliferation. The assessing step can be performed before or after the administering step.

The pharmaceutical compositions comprising an alkylester of α-methyl-DL-tyrosine (or salt thereof), for example, α-methyl-DL-tyrosine methyl ester hydrochloride, can further comprise a growth hormone inhibitor. The growth hormone can be pancreatic growth hormone. The growth hormone inhibitor can be octreotide or somatostatin.

The melanin promoter can be methoxsalen or melanotan II. The p450 3A4 promoter can be 5,5-diphenylhydantoin. The p450 3A4 promoter can be valproic acid or carbamazepine. The leucine aminopeptidase inhibitor can be N-[2S,3R)-3-amino-2-hydroxy-4-phenylbutyryl]-L-leucine or rapamycin. The pharmaceutical compositions of the invention can further comprise D-leucine.

Methods of treating cancer in a subject are also provided comprising administering an effective amount of an alkylester of α-methyl-DL-tyrosine (or salt thereof), for example, α-methyl-DL-tyrosine methyl ester hydrochloride, alone or in combination with one or more of the above-noted agents. In certain aspects, the α-methyl-DL-tyrosine methyl ester hydrochloride and at least two of the agents (e.g., melanin, promoters and/or inhibitors) are administered simultaneously. In other aspects, the alkylester of α-methyl-DL-tyrosine (or salt thereof), for example, α-methyl-DL-tyrosine methyl ester hydrochloride, and at least three of the agent are administered simultaneously. Each of the an alkylester of α-methyl-DL-tyrosine (or salt thereof), for example, α-methyl-DL-tyrosine methyl ester hydrochloride, and the agents can be administered simultaneously. The administration can be orally, subcutaneously, intravenously, transdermally, vaginally, rectally or in any combination thereof. The transdermal administration can be done with oleic acid, 1-methyl-2-pyrrolidone, or dodecylnonaoxyethylene glycol monoether. The alkylester of α-methyl-DL-tyrosine (or salt thereof), for example, α-methyl-DL-tyrosine methyl ester hydrochloride, and the agents can be administered during a cycle consisting of five to seven days of administering the components and one to two days of not administering. The alkylester of α-methyl-DL-tyrosine (or salt thereof), for example, α-methyl-DL-tyrosine methyl ester hydrochloride, and the agents can be administered over the course of at least six of said cycles. The melanin promoter can be methoxsalen. In another suitable method, 10 mg of the methoxsalen is administered orally and 0.25 mL of a 1 mg/mL suspension of the methoxsalen is administered subcutaneously. The melanin promoter can also be melanotan II. The p450 3A4 promoter can be 5,5-diphenylhydantoin. In another suitable method, 30 mg of the 5,5-diphenylhydantoin is administered orally. The p450 3A4 promoter can also be valproic acid or carbamazepine. The leucine aminopeptidase inhibitor can be N-[2S ,3R)-3-amino-2-hydroxy-4-phenylbutyryl]-L-leucine. In another suitable method, 20 mg of the N-[(2S,3R)-3-amino-2-hydroxy-4-phenylbutyryl]-L-leucine is administered orally. The leucine aminopeptidase inhibitor can also be rapamycin. The growth hormone can be pancreatic growth hormone. The growth hormone inhibitor can be octreotide. The method can further comprise administering an effective amount of D-leucine.

Methods of reducing cell proliferation in a subject are also provided comprising administering an effective amount of an alkylester of α-methyl-DL-tyrosine (or salt thereof), for example, α-methyl-DL-tyrosine methyl ester hydrochloride, alone or in combination with melanin and/or a melanin promoter; a p450 3A4 promoter; and a leucine aminopeptidase inhibitor to the subject in need thereof. The method of reducing cell proliferation can further comprise administration of a growth hormone inhibitor.

Representative methods of administration of the pharmaceutical compositions and combination therapies also are provided. Various aspects of the present invention further relate to methods of administering a pharmaceutical composition or combination therapy to a human patient for the treatment of cancer. The methods may comprise administering a pharmaceutical composition or combination therapy by generally accepted routes of administration (e.g., oral, subcutaneous, parenteral, inhalation, topical, etc.). In some instances, a pharmaceutical composition or combination therapy may be administered orally and/or subcutaneously. In some instances, a pharmaceutical composition or combination therapy may be administered to human patients between meals.

In certain instances of the present invention, a pharmaceutical composition or combination therapy may be administered to a human patient for 5 days per week for a period of 6 weeks, creating one cycle of 30 days of treatment. Depending on the outcome after 6 weeks or one cycle of treatment, additional cycles of the pharmaceutical composition or combination therapy may be administered.

In some embodiments, the disclosure is directed to the following aspects:
Aspect 1. A method for treating cancer in a patient comprising administering to the patient in need thereof a pharmaceutical composition comprising at least one alkylester of α-methyl-DL-tyrosine, or a pharmaceutically acceptable salt thereof.
Aspect 2. The method of aspect 1, wherein the alkyl ester is α-methyl-DL-tyrosine methyl ester.
Aspect 3. The method of aspect 1 or aspect 2, wherein the patient is administered at least one pharmaceutically acceptable salt of an alkylester of α-methyl-DL-tyrosine.
Aspect 4. The method of aspect 2, wherein the salt is α-methyl-DL-tyrosine methyl ester hydrochloride.
Aspect 5. The method of any one of the preceding aspects, further comprising administering to said patient at least one of:
   melanin, a melanin promoter that is methoxsalen or melanotan II, or a combination of melanin, methoxsalen and melanotan II;
   a p450 3A4 promoter that is 5,5-diphenylhydantoin, valproic acid, or carbamazepine; and
   a leucine aminopeptidase inhibitor that is N-[(2S,3R)-3-amino-2-hydroxy-4-phenylbutyryl]-L-leucine, or rapamycin.
Aspect 6. The method of any one of the preceding aspects, wherein said pharmaceutical composition is administered subcutaneously, intravenously, intramuscularly, or transdermally.
Aspect 7. The method of any one of the preceding aspects, wherein said pharmaceutical composition is an aqueous solution.
Aspect 8. The method of any one of the preceding aspects, wherein said cancer is non-small cell lung cancer, ovarian cancer, breast cancer, cervical cancer, pancreatic cancer, stomach cancer, brain cancer, liver cancer, testicular cancer, leukemia, lymphoma, appendix cancer, biliary cancer, choleangiocarcinoma, colon cancer, colorectal cancer, germ cell tumor, glioma, Hodgkin's lymphoma, lung cancer, neuroblastoma, prostate cancer, renal cancer, sarcoma, thyroid cancer, tongue cancer, tonsil squamous cell carcinoma, or urothelial cancer.
Aspect 9. The method of any one of the preceding aspects, further comprising administering to said patient an additional therapeutic agent for the treatment of cancer.

What is claimed:

1. A method for treating cancer in a patient comprising administering to the patient in need thereof a pharmaceutical composition comprising at least one alkylester of α-methyl-DL-tyrosine, or a pharmaceutically acceptable salt thereof.

2. The method of claim 1, wherein the alkyl ester is α-methyl-DL-tyrosine methyl ester.

3. The method of claim 1, wherein the patient is administered at least one pharmaceutically acceptable salt of an alkylester of α-methyl-DL-tyrosine.

4. The method of claim 3, wherein the salt is α-methyl-DL-tyrosine methyl ester hydrochloride.

5. The method of claim 1, further comprising administering to said patient at least one of:
melanin, a melanin promoter that is methoxsalen or melanotan II, or a combination of melanin, methoxsalen and melanotan II;
a p450 3A4 promoter that is 5,5-diphenylhydantoin, valproic acid, or carbamazepine; and
a leucine aminopeptidase inhibitor that is N-[(2S,3R)-3-amino-2-hydroxy-4-phenylbutyryl]-L-leucine, or rapamycin.

6. The method of claim 1, wherein said pharmaceutical composition is administered subcutaneously, intravenously, intramuscularly, or transdermally.

7. The method of claim 1, wherein said pharmaceutical composition is an aqueous solution.

8. The method of claim 1, wherein said cancer is non-small cell lung cancer, ovarian cancer, breast cancer, cervical cancer, pancreatic cancer, stomach cancer, brain cancer, liver cancer, testicular cancer, leukemia, lymphoma, appendix cancer, biliary cancer, choleangiocarcinoma, colon cancer, colorectal cancer, germ cell tumor, glioma, Hodgkin's lymphoma, lung cancer, neuroblastoma, prostate cancer, renal cancer, sarcoma, thyroid cancer, tongue cancer, tonsil squamous cell carcinoma, or urothelial cancer.

9. The method of claim 1, further comprising administering to said patient an additional therapeutic agent for the treatment of cancer.
